(19) United States
(12) Patent Application Publication
(10) Pub. No.: US 2007/0161542 A1
(43) Pub. Date: Jul. 12, 2007

(54) Ptb1b photoprobes

(76) Inventors: Chidambaram Ramachandran, Pierredfonds (CA); Michel Therien, Laval (CA); Cheuk K. Lau, Ile Bizard (CA); Robert Zamboni, Pointe Claire (CA)

Correspondence Address: MERCK AND CO., INC, P O BOX 2000, RAHWAY, NJ, 07065-0907, US

(21) Appl. No.: 10/582,467

(22) PCT Filed: Dec. 9, 2004
(86) PCT No.: PCT/CA04/02100

§ 371 Date: Jun. 12, 2006

Related U.S. Application Data
(60) Provisional application No. 60/529,257, filed on Dec. 12, 2003.

(51) Int. Cl. A61K 38/04; C07K 5/06

(52) U.S. Cl. 514/7; 548/957

(57) ABSTRACT

Photoaffinity probes (photoprobes) of the PTP1B enzyme contain the trifluoromethylphenyl diazirene photophore attached to compounds which bind to the PTP1B enzyme. The photoprobes bind irreversibly to the PTP1B enzyme upon irradiation, and can be used to study the binding of compounds to the PTP1B enzyme. Compounds that bind to the PTP1B enzyme may inhibit PTP1B and may be useful in the treatment of type 2 diabetes.

### FIELD OF THE INVENTION

This invention is directed to photoreactive compounds having an affinity for the PTP1B enzyme (photoprobes) that can be used to measure interactions of the PTP1B enzyme with the photoprobes and with compounds that may be therapeutically useful in the treatment of Type II diabetes.

### BACKGROUND OF THE INVENTION

One of the characteristic hallmarks of Type II diabetes is insulin resistance (1, 2). When insulin binds to the insulin receptor (IR), the receptor is autophosphorylated and leads to subsequent phosphorylation of intracellular substrates, resulting in glucose uptake. In Type II diabetes, the insulin is less effective for a variety of known and unknown reasons (3). Protein tyrosine phosphatase 1B (PTP1B) has been suggested to be the phosphatase responsible for dephosphorylating the insulin receptor, resulting in down-regulation of the IR kinase activity. It has been proposed that inhibition of PTP1B may be a viable drug therapy for Type II diabetes by improving insulin receptor signaling. Evidence for the involvement of PTP1B in the insulin pathway includes both in vivo (4, 5) and in vitro (6, 7, 8) observations of an interaction between PTP1B and the IR. Other evidence includes osmotic loading of neutralizing antibodies to inhibit PTP1B activity, leading to enhanced insulin signaling at the level of the insulin receptor itself (9), whereas overexpression of PTP1B in transfected cells causes inhibition of IR and IRS-1 phosphorylation (10). Furthermore, PTP1B knockout mice exhibit increased insulin sensitivity and decreased susceptibility to diet-induced obesity compared with wild-type mice, supporting the hypothesis that PTP1B is involved in the insulin pathway (11).

Several small molecule inhibitors for PTP1B have been described (12, 13, 14, 15). Some of these have been reported to function in vivo in diabetic mice (16, 17). The majority of inhibitors reported for PTP1B are non-selective, have poor bioavailability, and are not cell-permeable, as measured by CaCo-2 cell permeability assays. The determination of whether compounds inhibit PTP1B in a cell has often been based on downstream events, such as assays of IR, Akt, and MAPK phosphorylation levels or assays of glucose uptake. However, these assays do not directly measure the binding of the compound to PTP1B. Several tools can be used to measure enzyme occupancy by an inhibitor in a cell. These include competitive binding of the enzyme with a selective substrate and the measurement of free and bound enzyme in competitive binding studies with a potent inhibitor that binds tightly or irreversibly. Both the selective substrate and the irreversible inhibitor should possess a suitable tag for detection and should be cell permeable and selective for the enzyme of interest to be useable in a cell-based system. The phosphatase substrates for PTP1B are not selective and are readily hydrolyzed by other phosphatases. There are known irreversible inhibitors of PTP1B, such as iodoacetic acid and other thiol-labeling reagents, but these are also non-selective.

An approach to developing an irreversible selective inhibitor to label PTP1B is the use of photoaffinity labeling. A photolabile moiety can be attached to a known potent inhibitor of the enzyme of interest to yield a photoaffinity reagent (“photoprobe”). Upon irradiation of the photoprobe in the presence of the enzyme, the photoprobe becomes irreversibly bound to the enzyme, so that the enzyme has become labeled by the photoprobe. Photoaffinity labeling has the advantage that the ligand is chemically inert until irradiation, thereby allowing transport into a cell and binding to the enzyme or receptor of interest before generation of a highly reactive intermediate (20). Several photolabile moieties (photophores) are known that may be suitable for incorporation into an inhibitor, including acrylazides, benzophenones, and trifluoromethylphenyl diazirene photophores. Each of these has different advantages and disadvantages. The acrylazides require UV irradiation; the UV light may destroy cells and enzymes. Benzophenones are excellent photophores, as they are activated at longer wavelengths that do not destroy the cells and enzymes, and the radicals that are produced are selective for C—H bonds rather than the OH bonds of water (20). However, a benzophenone reagent can significantly change the lipophilicity of the compound that is being labeled and can produce a substantial structural change in the inhibitor, resulting in decreased potency towards the target of interest (21). Trifluoromethylphenyl diazirenes are activated at 350-360 nm, as are the benzophenones, so that irradiation with UV light is avoided, thereby minimizing or avoiding damage to the cells and enzymes. However, the carbene that results from irradiation of the trifluoromethylphenyl diazirenes is electrophilic, which can cause the carbene to be relatively more reactive with water than with C—H bonds (22).

### SUMMARY OF THE INVENTION

A class of photoprobes of the PTP1B enzyme is described herein which can be used to study or measure the interactions of PTP1B with compounds that may act as inhibitors of the PTP1B enzyme in biologic systems, such as cells. The photoaffinity probes described below contain the trifluoromethylphenyl diazirene photophore. The photoprobes can be used efficiently in aqueous systems (cells), even though the carbene intermediate generated by irradiation of the photophore is expected to be reactive with water as well as with CH bonds. The trifluoromethylphenyl diazirene photophore apparently does not cause a significant change in the inhibitor structure or its lipophilicity.

Biochemical and photochemical interactions of the photoprobes have been observed with purified PTP1B and with intracellular PTP1B in HEPG2 whole and lysed cells. Enzyme occupancy was detected for several phosphatase inhibitors in HEPG2 whole cells, indicating that the compounds are cell permeable and can bind to PTP1B in a whole cell.

The photoprobes are compounds having the formula I, including salts thereof: 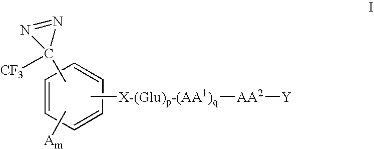
wherein Glu is a glutamic acid residue in which the gamma-carboxy group is a free carboxylic acid or a C₁₋₃ alkyl ester;
AA¹ is an amino acid residue having the formula 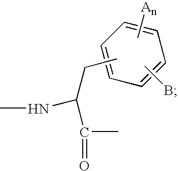
AA² is an amino acid residue having the formula 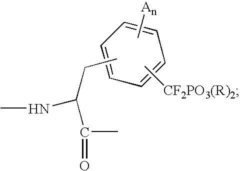
X is a difunctional group selected from CH₂ and carbonyl;
Y is selected from the group consisting of OH and NR₂;
Each R is independently selected from the group consisting of H and C₁₋₆ alkyl;
Each A is a substituent selected from the group consisting of CH₃, CF₃, and halogen;
Each B is a substituent selected from the group consisting of H, —CF₂PO₃(R)₂ and 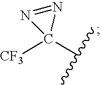
m is 0, 1, or 2;
each n is independently 0, 1, or 2;
p is 0, 1, or 2; and
q is 0, 1, or 2.

The compounds of this invention include salts and esters, as well as the free acids, of the carboxylic acid and phosphonic acid groups in the compounds of formula I. For in vivo studies, such salts are pharmaceutically acceptable salts. Alkyl groups can be either linear or branched, unless otherwise stated.

The divalent amino acid residues Glu, AA¹, and AA² are bonded to each other and to X and Y through the amino groups and the carboxy groups following the convention that the amino group of each amino acid residue is on the left and the carboxy group of each amino acid residue is on the right.

### DETAILED DESCRIPTION OF THE INVENTION

In many preferred subgroups of the above compounds, m is 0 or 1.

In many preferred subgroups of the above compounds, n is 0 or 1.

In many preferred subgroups of the above compounds, p is 0 or 1.

In many preferred subgroups of the above compounds, q is 0 or 1.

In many preferred subgroups of the above compounds, Glu is a glutamic acid residue, where the gamma carboxylic acid group is a free acid or methyl ester, including salts of the carboxylic acid group.

In many preferred subgroups of the above compounds, Glu is a glutamic acid residue, where the gamma carboxylic acid group is a free acid or a salt thereof.

In a subgroup of preferred compounds of Formula I, Glu is a glutamic acid residue, where the gamma carboxylic acid group is a free carboxylic acid or a methyl ester;

m is 0 or 1;

each n is independently 0 or 1;

p is 0 or 1; and

q is 0 or 1.

In many preferred compounds, Y is NR₂, and generally, Y is NH₂.

In another embodiment of the compounds of Formula I or of the compounds described above:

Each A is a halogen independently selected from F, Cl, Br, and I;

Each group B is a substituent group selected from the groups —CF₂PO₃H₂ and

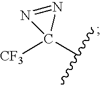

R is H; and

q is 0 or 1.

In other embodiments of the compounds described above, AA¹ and AA² are each phenylalanine residues (i.e., they have the stereochemistry of phenylalanine), which have substituents on the phenyl ring of the phenylalanine as defined above.

In other embodiments of the compounds described above, AA¹ and AA² are amino acid residues having the formula

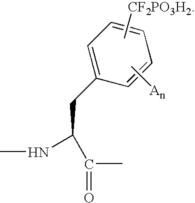

In these compounds, each A is independently selected from either Br or I;

m is 0 or 1;

each n is independently 0 or 1;

p is 0 or 1; and

q is 0 or 1.

In a preferred subgroup of the compounds defined above, A is Br and n is 1.

In other embodiments of the compounds having formula I as described above, AA¹ is an amino acid residue having the formula

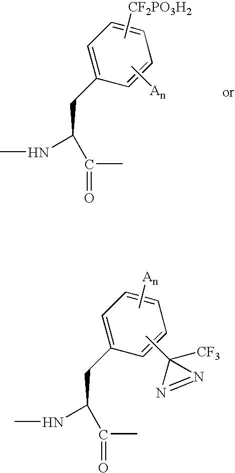

Glu is a glutamic acid residue in which the the gamma-carboxy group of the glutamic acid residue is a free carboxylic acid or methyl ester;

AA² is a phenylalanine residue of the formula

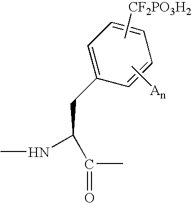

Each A is independently selected from Br and I;

Y is —NH₂;

m is 0 or 1;

each n is independently 0 or 1;

p is 0 or 1; and

q is 0 or 1.

Subgroups of the compounds defined above include compounds in which the gamma-carboxy group of Glu is a free carboxylic acid, including salts. Subgroups of the compounds defined above include compounds in which A is Br or I, m is 0 or 1, and n is 1, except that in AA¹ and AA², A is Br.

Specific compounds of this invention are provided in the Table of Compounds below. The compounds and methods of making and using the compounds are described in detail in the examples below.

Table of Compounds

| | |
|---|---|
| Example 1 | 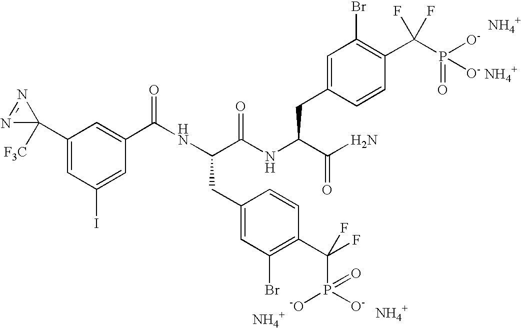 |
| Example 2 | 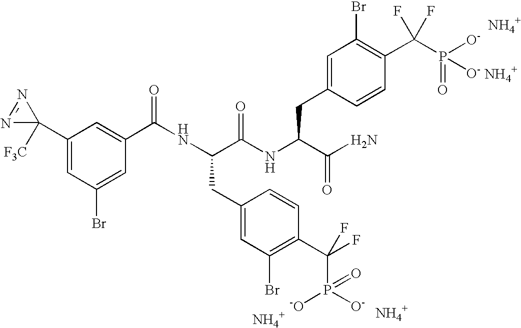 |
| Example 3 | 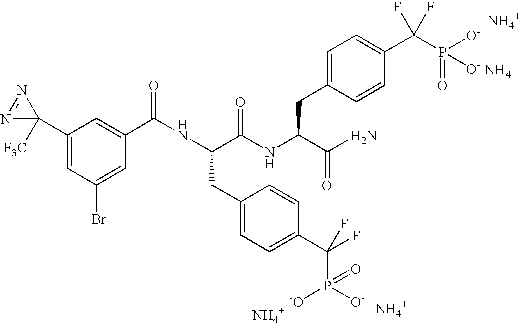 |
| Example 4 | 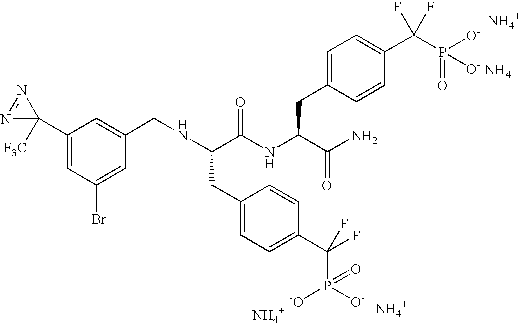 |
| Example 5 | 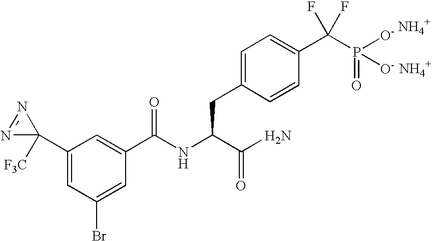 |
| Example 6 | 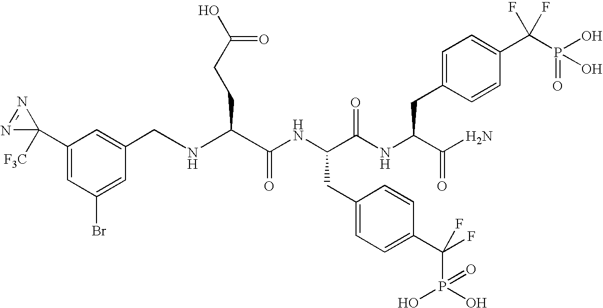 |
| Example 7 | 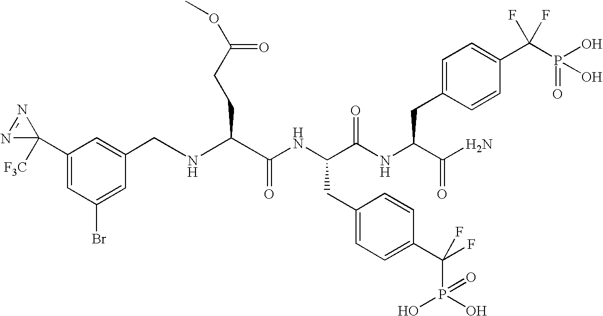 |
| Example 8 | 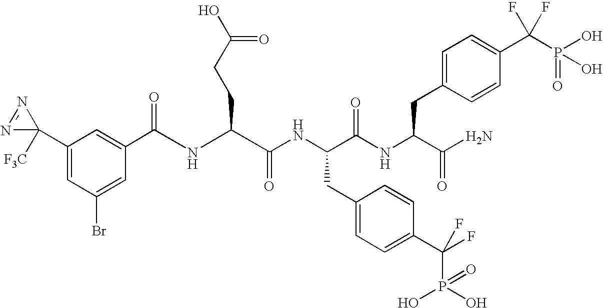 |
| Example 9 | 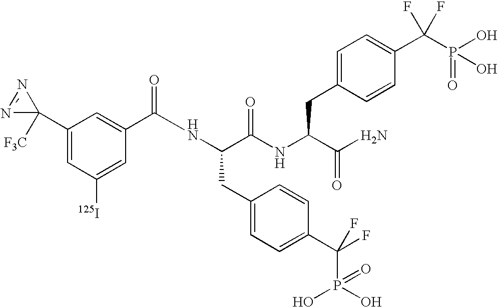 |
| Example 10 | 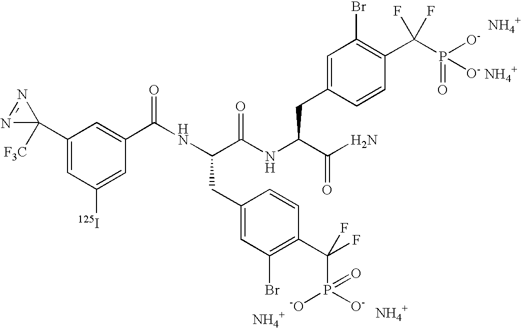 |
| Example 11 | 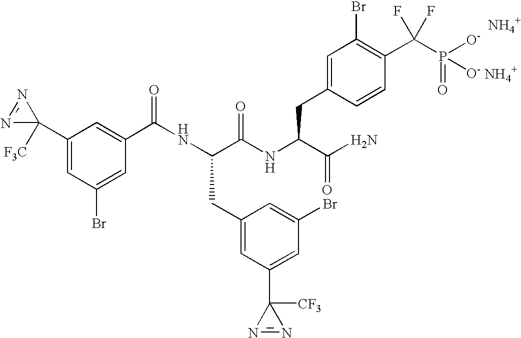 |

The photoprobes of this invention bind reversibly to the PTP1B enzyme before irradiation. Upon irradiation, the compounds irreversibly bind to PTP1B at the site where the photoprobes were previously reversibly bound, resulting in PTP1B that is labeled in the ligand binding domain of the enzyme. The photoprobes may also be labeled with radioactive isotopes to yield radiolabeled PTP-1B for studies of PTP1B activity in cell media and at physiological concentrations.

### EXAMPLES

The following examples are provided to illustrate embodiments of the invention. These examples should not be construed as limiting the scope of the invention in any way.

### Example 1

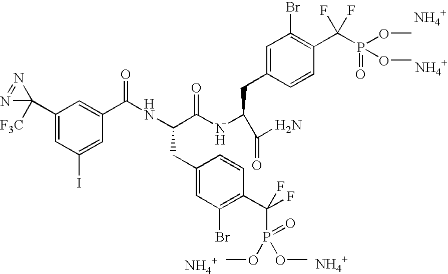

### Tetraamnionium 3-bromo-4-(difluoro(phosphonato)methyl)-N-(3-iodo-5-(3-(trifluoromethyl)-3H-diaziren-3-yl)benzoyl)-L-phenylalanyl-3-bromo-4-(difluoro(phosphonato)methyl)-L-phenylalaninamide

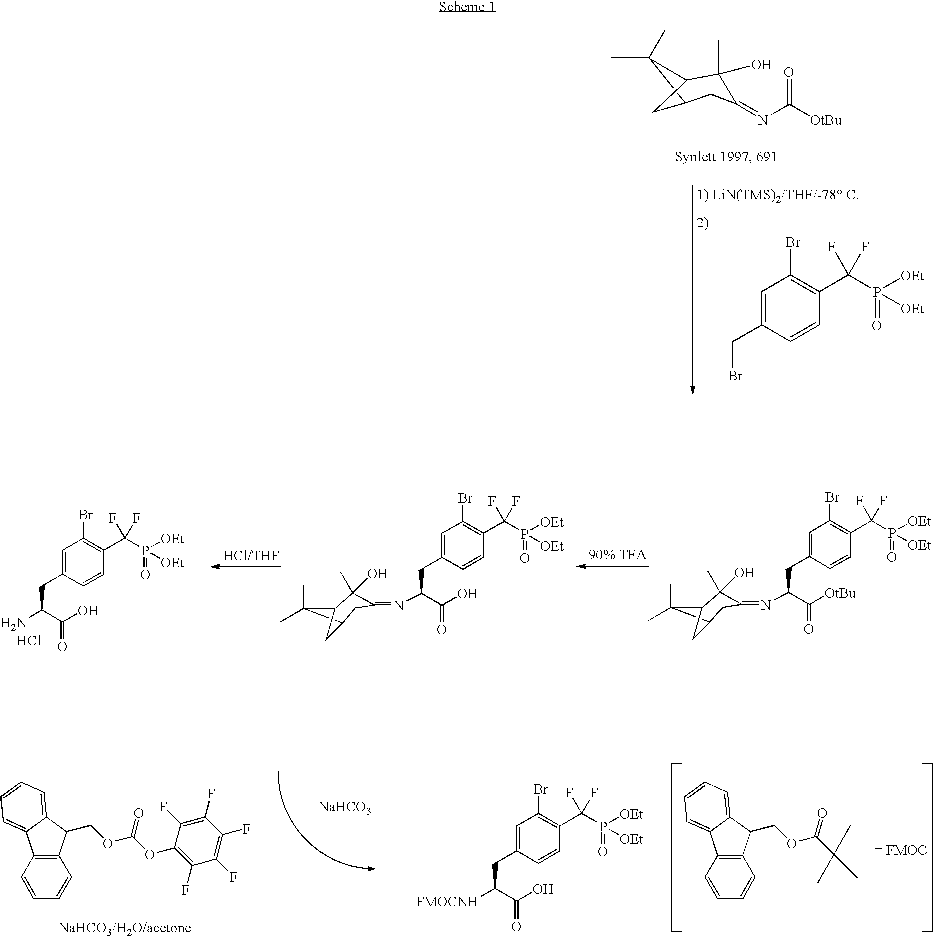

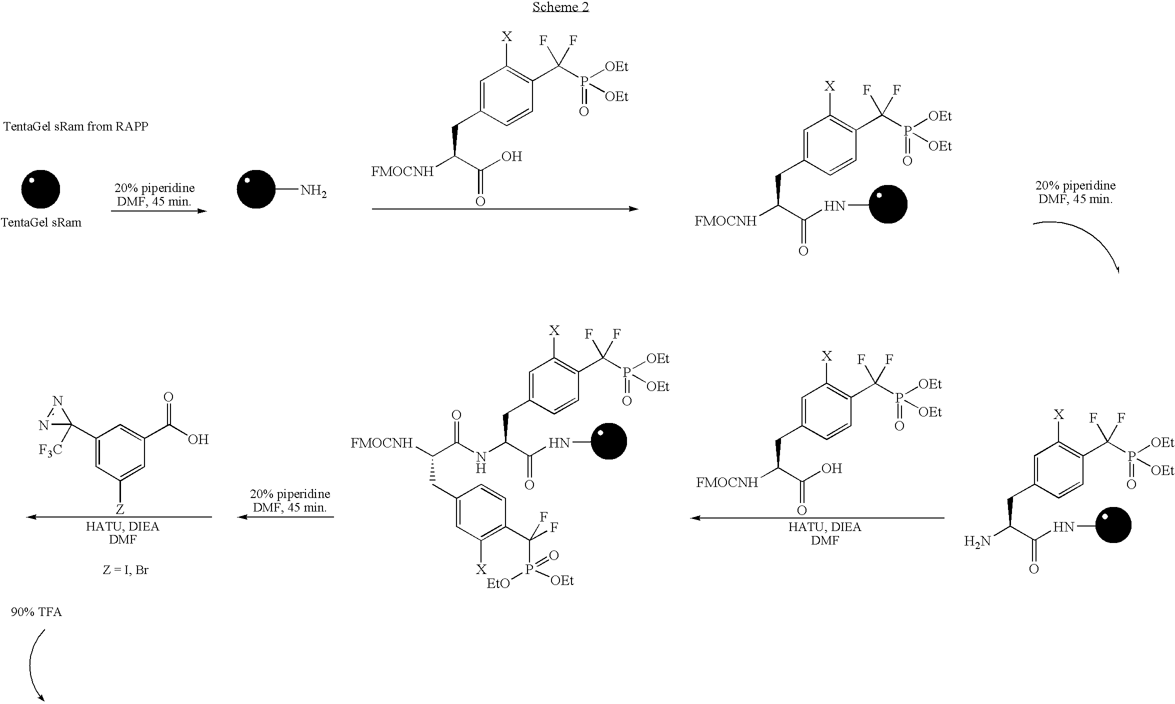

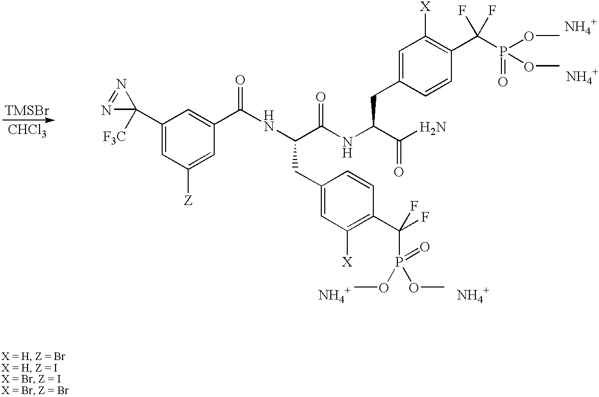

### Steps 1-4: See Scheme 1

### Step 1: Diethyl (2-bromo-4-((2S)-2-(((3Z)-2-hydroxy-2,6,6-trimethylbicyclo[3.1.1]hept-3-ylidene)amino)-3-oxobutyl)phenyl)(difluoro)methylphosphonate

For the preparation and use of of tert-butyl N-(2-hydroxy-2,6,6-trimethylbicyclo[3.1.1]hept-3-ylidene)glycinate, see M. Jacob et al., Synlett, 1997, pp. 691-692. To a solution of tert-butyl N-(2-hydroxy-2,6,6-trimethylbicyclo[3.1.1]hept-3-ylidene)glycinate (1.124 g) in THF (30 mL) at −78° C. was added dropwise lithium hexamethyldisilazide (7.5 mL) and the mixture was stirred at −78° C. for 15 minutes. A solution of diethyl (2-bromo-4-(bromomethyl)phenyl)(difluoro)methylphosphonate (1.57 g) in THF was then added and the mixture was stirred at −78° for 3 hrs. A saturated solution of ammonium chloride was added and the mixture was extracted with ethyl acetate. The extract was dried over magnesium sulfate and filtered, and the solvent was evaporated under vacuum. Purification by silica gel chromatography using ethyl acetate and hexane as eluent afforded diethyl (2-bromo-4-((2S)-2-(((3Z)-2-hydroxy-2,6,6-trimethylbicyclo[3.1.1]hept-3-ylidene)amino)-3-oxobutyl)phenyl)(difluoro)methylphosphonate.

### Step 2: 3-bromo-4-((diethoxyphosphoryl)(difluoro)methyl)-N-(2-hydroxy-2,6,6-trimethylbicyclo[3.1.1]hept-3-ylidene)-L-phenylalanine

To diethyl (2-bromo-4-((2S)-2-(((3Z)-2-hydroxy-2,6,6-trimethylbicyclo[3.1.1 ]hept-3-ylidene)amino)-3-oxobutyl)phenyl)(difluoro)methylphosphonate (1.7 g) was added trifluoroacetic acid (10 mL) and water (1 mL). The mixture was stirred at room temperature overnight. Evaporation of the solvent under vacuum afforded the title product.

### Step 3: 3-bromo-4-((diethoxyphosphoryl)(difluoro)methyl)-L-phenylalanine hydrochloride

The product from the previous step was dissolved in THF (15 mL), and 1N HCL (15 mL) was added. The mixture was stirred at room temperature for 3 days, the solvent was then evaporated under vacuum and coevaporated with ethanol several times under vacuum. The residue was dried under high vacuum, then triturated in diethyl ether to afford 3-bromo-4-((diethoxyphosphoryl)(difluoro)methyl)-L-phenylalanine hydrochloride.

### Step 4: 3-bromo-4-((diethoxyphosphoryl)(difluoro)methyl)-N-((9H-fluoren-9-ylmethoxy)carbonyl)-L-phenylalanine

To a solution of 3-bromo-4-((diethoxyphosphoryl)(difluoro)methyl)-L-phenylalanine hydrochloride (88 mg) in water (1.5 mL) was added sodium bicarbonate (23 mg) followed by a solution of 9H-fluoren-9-ylmethyl pentafluorophenyl carbonate (96 mg) in dioxane (1.5 mL) and acetone (2 mL). The resulting solution was stirred at room temperature overnight. The solvent was evaporated under vacuum, and the residue was washed with diethyl ether (2×), acidified with 6 N HCl, and extracted with ethyl acetate. Purification by silica gel chromatography afforded the title compound.

### Steps 5-12: See Scheme 2.

### Step 5:

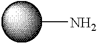 NH₂

1 g of TentaGel® S RAM resin (RAPP polymer, ˜0.2 mmol/g) as represented by the shaded bead in Scheme 2 was treated with piperidine (0.8 mL) in DMF (3.2 mL) for 30 minutes. The resin was then washed with DMF (5×) and methylene chloride (5×), flushed with nitrogen and pumped dry under high vacuum.

### Step 6:

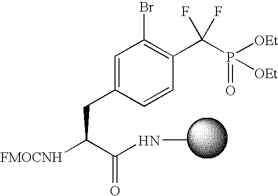

To a solution of 3-bromo-4-((diethoxyphosphoryl)(difluoro)methyl)-N-((9H-fluoren-9-ylmethoxy)carbonyl)-L-phenylalanine (449 mg) and O-(7-azabenzotriazol-1-yl)-N,N,N′,N′-tetramethyluronium hexafluorophosphate (HATU) (305 mg) in DMF was added diisopropylethylamine (288 μL), and the mixture was stirred at room temperature for 15 minutes. The mixture was then added to the resin from the previous step and shaken for 3 hrs. The resin was then washed with DMF (5×) and methylene chloride (5×), flushed with nitrogen and pumped dry under high vacuum.

### Step 7:

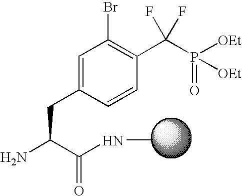

The compound of step 6 was treated as in step 5.

### Step 8:

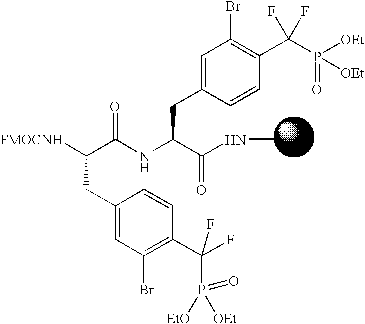

The compound of step 7 was treated as in step 6.

### Step 9:

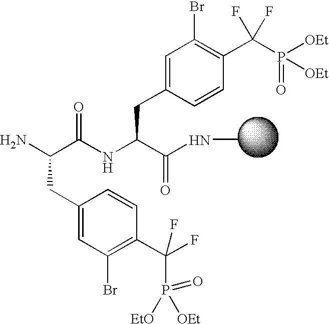

The compound of step 8 was treated as in step 5.

### Step 10:

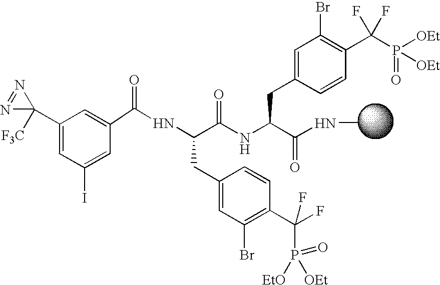

The compound of step 9 was treated as in step 6, but using 3-iodo-5-(3-(trifluoromethyl)-3H-diaziren-3-yl)benzoic acid instead of 3-bromo-4-((diethoxyphosphoryl)(difluoro)methyl)-N-((9H-fluoren-9-ylmethoxy)carbonyl)-L-phenylalanine.

### Step 11:

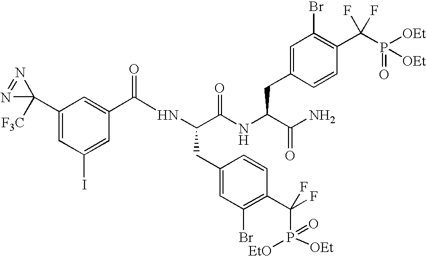

The compound of step 10 (200 mg of resin) was treated with trifluoroacetic acid (1.8 mL) and water (0.2 mL) for 45 minutes. Then the solvent was recovered. The resin was washed with another portion of trifluoroacetic acid (1.8 mL) and water (0.2 mL), the 2 portions were combined, and the solvent was evaporated under vacuum.

### Step 12:

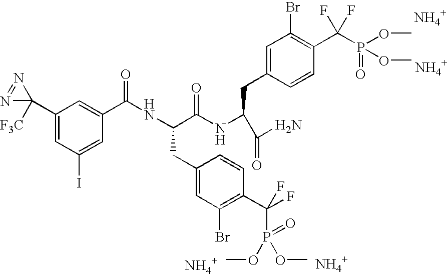

### Tetraammonium 3-bromo-4-(difluoro(phosphonato)methyl)-N-(3-iodo-5-(3(trifluoromethyl)-3H-diaziren-3-yl)benzoyl)-L-phenylalanyl-3-bromo-4-(difluoro(phosphonato)methyl)-L-phenylalaninamide

To the compound of step 11 (5 mg) in chloroform (2 mL) was added bromotrimethylsilane (1 mL), and the mixture was stirred overnight. The solvent was evaporated under vacuum, coevaporated with chloroform (3×), and coevaporated with a methanol/water (85/15) mixture. The residue was purified by HPLC using a C₁₈ column with methanol and aqueous ammonium acetate buffer as solvent to afford the title compound.

M.S. (ESI) m/z 1065.8 (M−H)⁻.

### Example 2

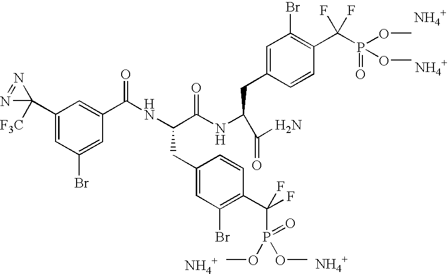

### Tetraammonium 3-bromo-4-(difluoro(phosphonato)methyl)-N-(3-bromo-5-(3-(trifluoromethyl)-3H-diaziren-3-yl)benzoyl)-L-phenylalanyl-3-bromo-4-(difluoro(phosphonato)methyl)-L-phenylalaninamide

Using the same procedure as in example 1, but using 3-bromo-5-(3-(trifluoromethyl)-3H-diaziren-3-yl)benzoic acid instead of 3-iodo-5-(3-(trifluoromethyl)-3H-diaziren-3-yl)benzoic acid in step 10, the title compound was obtained.

M.S. (ESI) m/z 1020.1 (M−H)⁻.

### Example 3

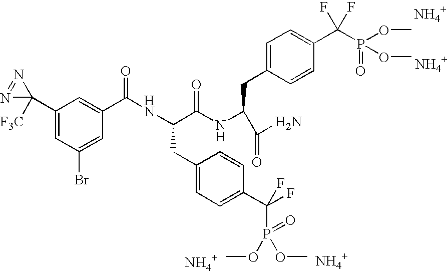

### Tetraammonium N-(3-bromo-5-(3-(trifluoromethyl)-3H-diaziren-3-yl)benzoyl)-4-(difluoro(phosphonato)methyl)-L-phenylalanyl-4-(difluoro(phosphonato)methyl)-L-phenylalaninamide

Using the same procedure as in example 1, steps 5 to 12, but using 4-((diethoxyphosphoryl)(difluoro)methyl)-N-((9H-fluoren-9-ylmethoxy)carbonyl)-L-phenylalanine instead of 3-bromo-4-((diethoxyphosphoryl)(difluoro)methyl)-N-((9H-fluoren-9-ylmethoxy)carbonyl)-L-phenylalanine in steps 6 and 8, and using 3-bromo-5-(3-(trifluoromethyl)-3H-diaziren-3-yl)benzoic acid instead of 3-iodo-5-(3-(trifluoromethyl)-3H-diaziren-3-yl)benzoic acid in step 10, the title compound was obtained.

M.S. (ESI) m/z 861 (M−H)⁻.

### Example 4

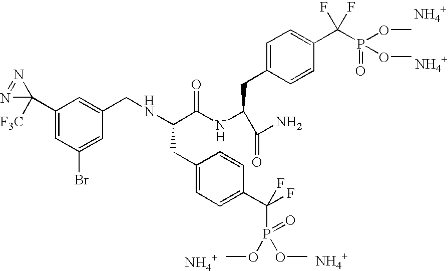

### Tetraammonium N-(3-bromo-5-(3-(trifluoromethyl)-3H-diaziren-3-yl)benzyl)-4-(difluoro(phosphonato)methyl)-L-phenylalanyl-4-(difluoro(phosphonato)methyl)-L-phenylalaninamide

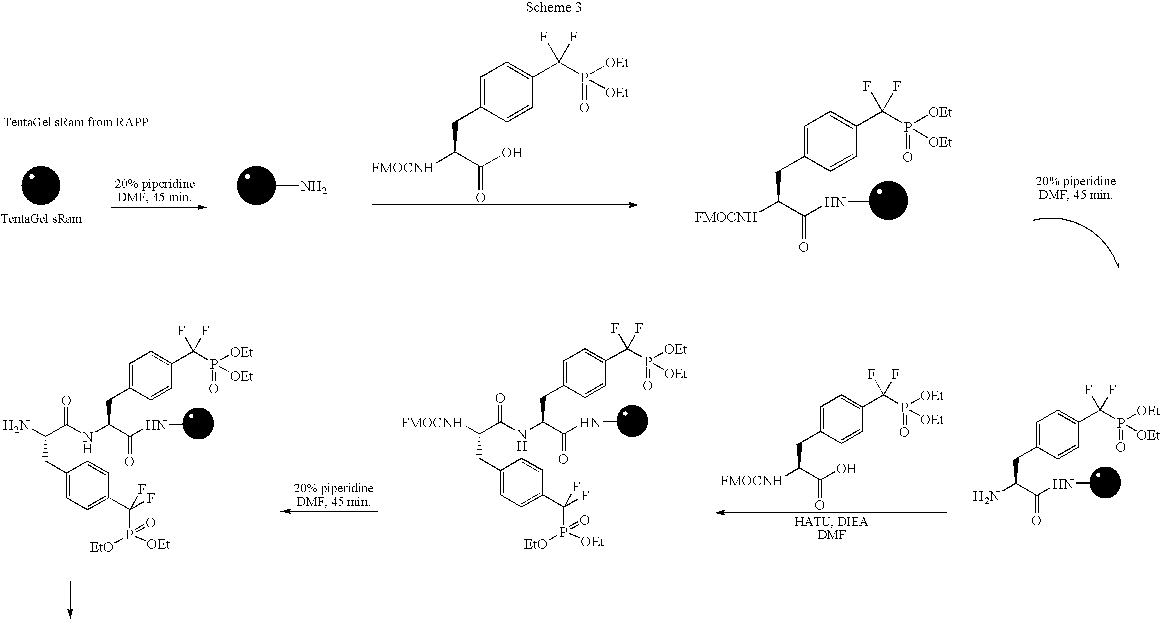

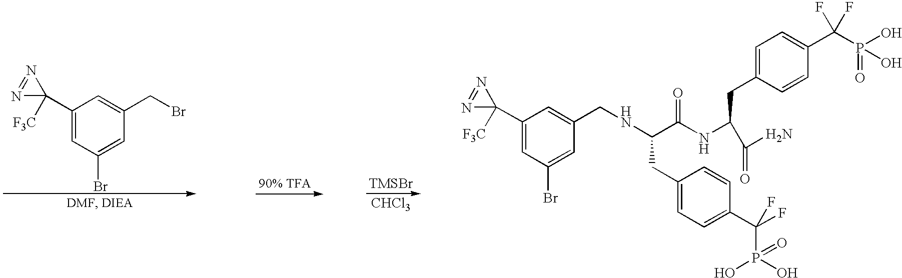

See Scheme 3. The same procedure was used as in example 1, steps 5 to 12, but 4-((diethoxyphosphoryl)(difluoro)methyl)-N-((9H-fluoren-9-ylmethoxy)carbonyl)-L-phenylalanine was used in steps 6 and 8 instead of 3-bromo-4-((diethoxyphosphoryl)(difluoro)methyl)-N-((9H-fluoren-9-ylmethoxy)carbonyl)-L-phenylalanine, and 3-bromo-5-(3-(trifluoromethyl)-3H-diaziren-3-yl)benzyl bromide and diisopropylethylamine (7.5 eq) were used instead of 3-iodo-5-(3-(trifluoromethyl)-3H-diaziren-3-yl)benzoic acid and HATU in step 10 to yield the title compound.

M.S. (ESI) m/z 848.3 (M−H)⁻.

### Example 5

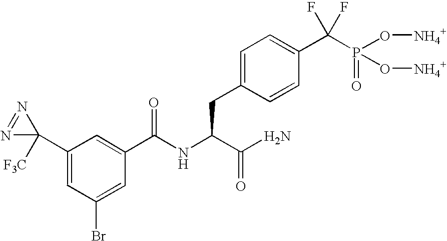

### Diammonium N-(3-bromo-5-(3-(trifluoromethyl)-3H-diaziren-3-yl)benzoyl)-4-(difluoro(phosphonato)methyl)-L-phenylalaninamide

The same procedure was followed as in example 1, steps 5 to 7, but 4-((diethoxyphosphoryl)(difluoro)methyl)-N-((9H-fluoren-9-ylmethoxy)carbonyl)-L-phenylalanine was used instead of 3-bromo-4-((diethoxyphosphoryl)(difluoro)methyl)-N-((9H-fluoren-9-ylmethoxy)carbonyl)-L-phenylalanine in step 6. Steps 8 and 9 were skipped. Step 10 was followed using 3-bromo-5-(3-(trifluoromethyl)-3H-diaziren-3-yl)benzoic acid instead of 3-iodo-5-(3-(trifluoromethyl)-3H-diaziren-3-yl)benzoic acid. Steps 11 and 12 were performed as in Example 1 to afford the title compound.

M.S. (ESI) m/z 584.8 (M−H)⁻.

### Example 6

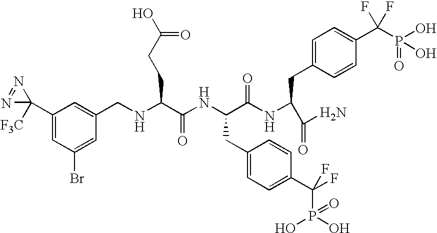

### N-(3-bromo-5-(3-(trifluoromethyl)-3H-diaziren-3-yl)benzyl)-L-α-glutamyl-4-(difluoro(phosphono)methyl)-L-phenylalanyl-4-(difluoro(phosphono)methyl)-L-phenylalaninamide

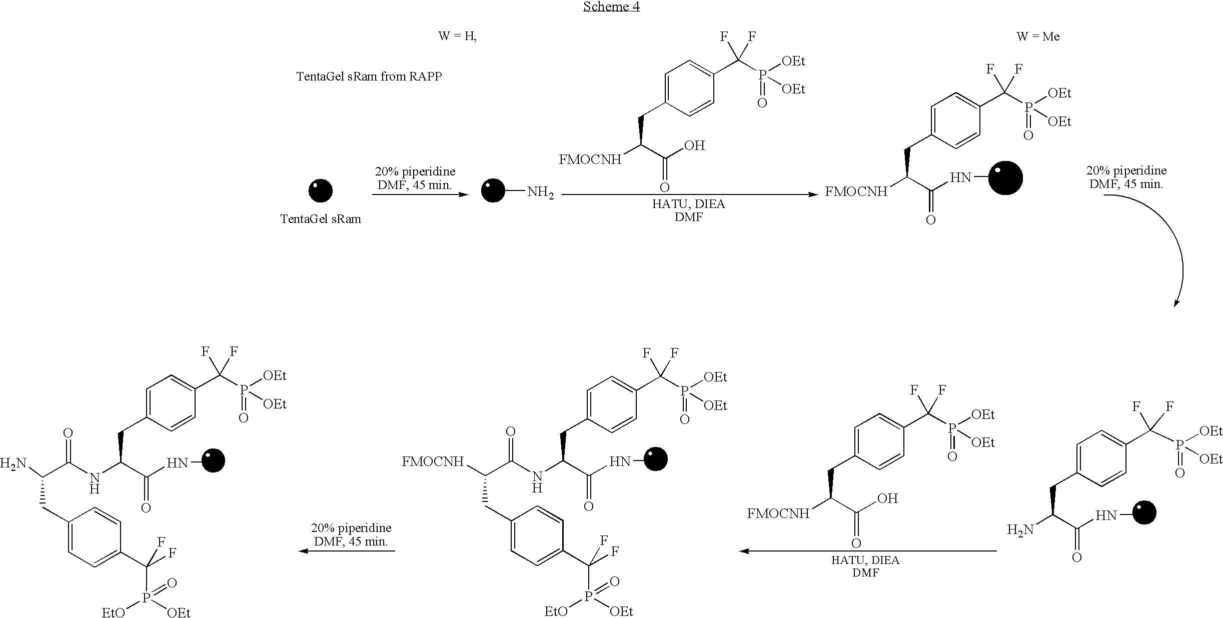

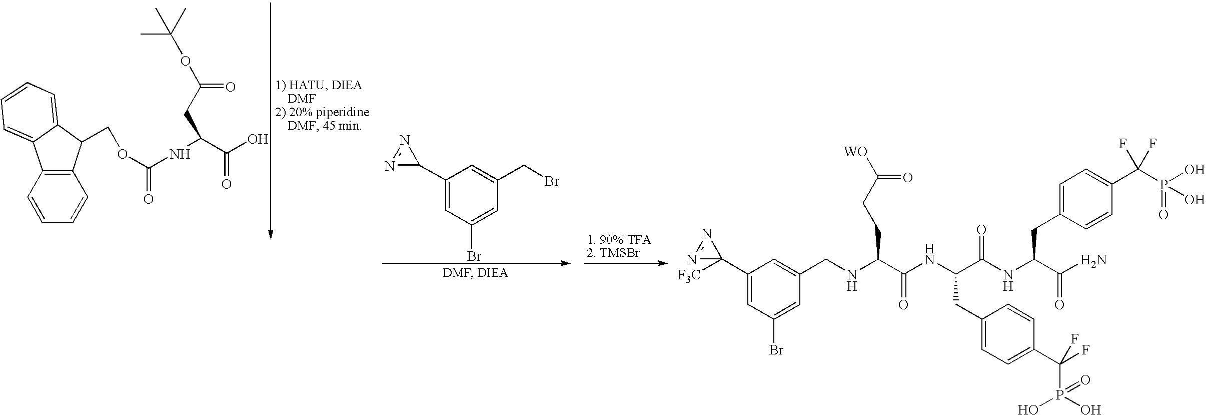

See Scheme 4. The same procedure was followed as in example 1, steps 5 to 9, but 4-((diethoxyphosphoryl)(difluoro)methyl-N-((9H-fluoren-9-ylmethoxy)carbonyl)-L-phenylalanine was used instead of 3-bromo-4-((diethoxyphosphoryl)(difluoro)methyl)-N-((9H-fluoren-9-ylmethoxy)carbonyl)-L-phenylalanine in steps 6 and 8 (X═H in Scheme 2). The product obtained after treatment with piperidine and DMF using the method of step 9 of Example 1 was reacted with N-((9H-fluoren-9-ylmethoxy)carbonyl)-L-glutamic acid gamma-t-butyl ester instead of 3-bromo-4-((diethoxyphosphoryl)(difluoro)methyl)-N-((9H-fluoren-9-ylmethoxy)carbonyl)-L-phenylalanine using the methods of steps 6 and 7 of Example 1. That product in turn was reacted with 3-bromo-5-(3-(trifluoromethyl)-3H-diaziren-3-yl)benzyl bromide and diisopropylethylamine (7.5 eq) instead of 3-iodo-5-(3-(trifluoromethyl)-3H-diaziren-3-yl)benzoic acid and HATU using the method of step 10 of Example 1. After treatment with trifluoroacetic acid as described in Step 11 and bromotrimethylsilane as in Step 12 of Example 1 followed by HPLC purification using a C₁₈ column with methanol and aqueous ammonium acetate buffer as solvent, the title compound was obtained. The reagents and intermediate products are shown in Scheme 4.

M.S. (ESI) m/z 976.9 (M−H)⁻.

### Example 7

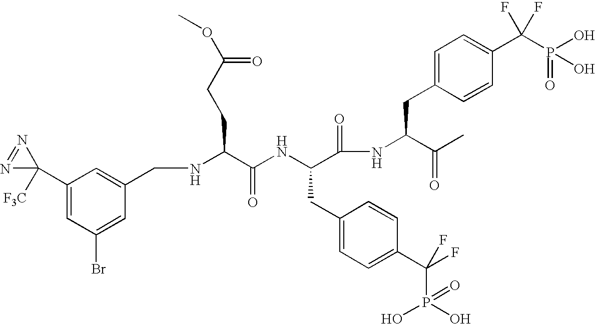

Using the same procedure as in Example 6 (Scheme 4), the methyl glutamate derivative shown above was obtained. The free acid and methyl ester compounds of Examples 6 and 7 are both produced by the same method, the esterification of compound 6 to compound 7 occuring during the HPLC purification by reaction with the methanol used as a solvent, and elute separately during the HPLC purification. M.S. (ESI) m/z 991 (M−H)⁻.

### Example 8

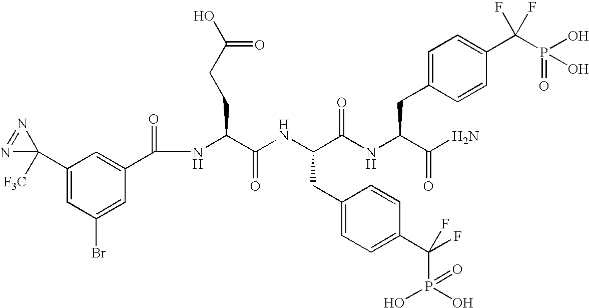

### N-(3-bromo-5-(3-(trifluoromethyl)-3H-diaziren-3-yl)benzoyl)-L-α-glutamyl-4-(difluoro(phosphono)methyl)-L-phenylalanyl-4-(difluoro(phosphono)methyl)-L-phenylalaninamide

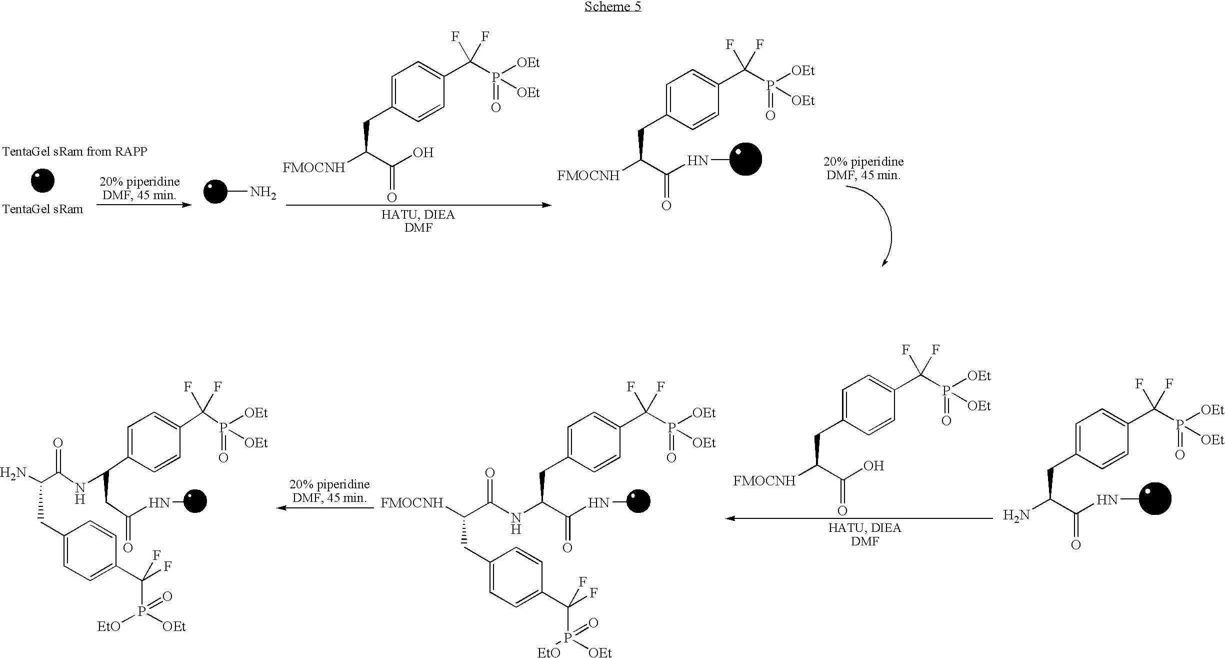

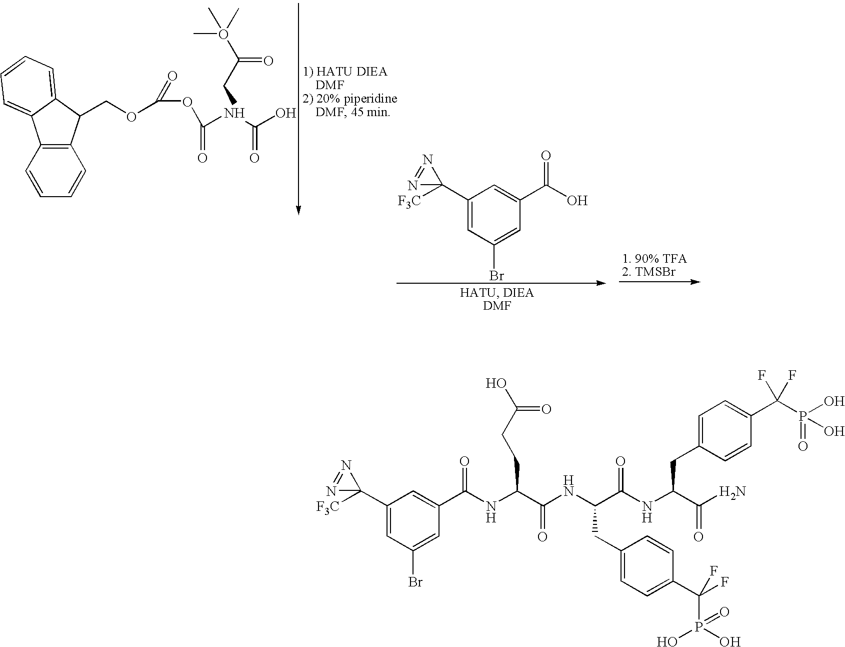

See Scheme 5. The same procedure was followed as in example 1, steps 5 to 9, but 4-((diethoxyphosphoryl)(difluoro)methyl)-N-((9H-fluoren-9-ylmethoxy)carbonyl)-L-phenylalanine was used instead of 3-bromo-4-((diethoxyphosphoryl)(difluoro)methyl)-N-((9H-fluoren-9-ylmethoxy)carbonyl)-L-phenylalanine in steps 6 and 8 (X═H in Scheme 2). The product that was obtained after treatment with piperidine and DMF as in Ex. 1, step 9, was reacted with N-((9H-fluoren-9-ylmethoxy)carbonyl)-L-glutamic acid gamma-t-butyl ester instead of 3-bromo-4-((diethoxyphosphoryl)(difluoro)methyl)-N-((9H-fluoren-9-ylmethoxy)carbonyl)-L-phenylalanine using the method of steps 6 and 7 of Example 1. That product in turn was reacted with 3-bromo-5-(3-(trifluoromethyl)-3H-diaziren-3-yl)benzoic acid instead of 3-iodo-5-(3-(trifluoromethyl)-3H-diaziren-3-yl)benzoic acid using the method of step 10 in Example 1. After following the method of steps 11 and 12 of Scheme 1, the title compound was obtained.

M.S. (ESI) m/z 976.9 (M−H)⁻.

### Example 9

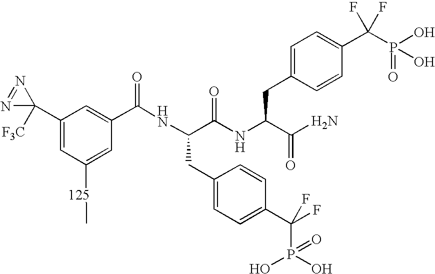

### 4-(Difluoro(phosphono)methyl)-N-(3-iodo-5-(3-(trifluoromethyl)-3H-diaziren-3-yl)benzoyl)-L-phenylalanyl-4-(difluoro(phosphono)methyl)-L-phenylalaninamide

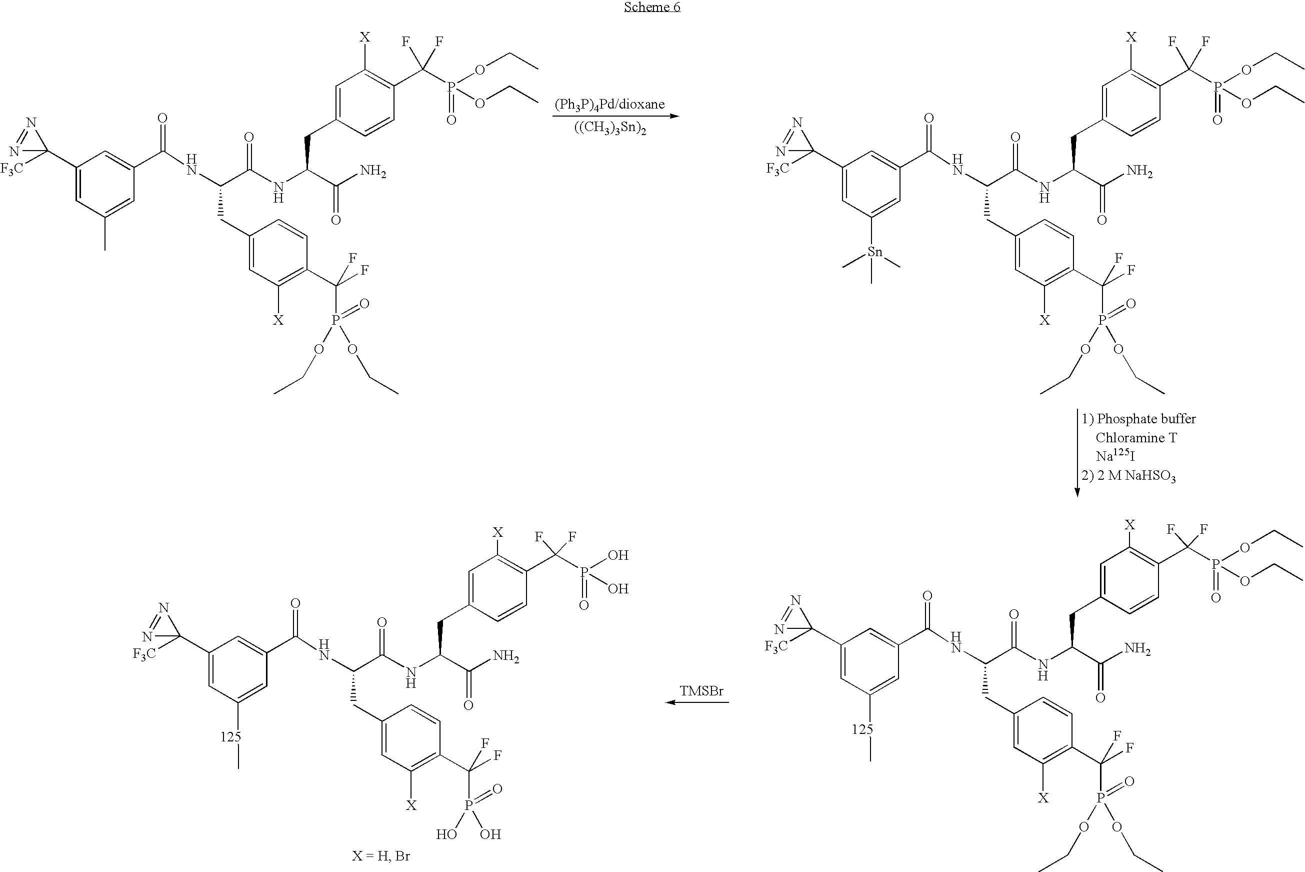

The three steps shown in Scheme 6 for X═H are described as steps 2, 3, and 4 below. The synthesis of the starting material in Scheme 6 is described in Step 1 below:

Step 1: The same procedure was followed as in example 1, steps 5 to 11, but using 4-((diethoxyphosphoryl)(difluoro)methyl-N-((9H-fluoren-90ylmethoxy)carbonyl-L-phenylalanine instead of 3-bromo-4-((diethoxyphosphoryl)(difluoro)methyl)-N-((9H-fluoren-9-ylmethoxy)carbonyl)-L-phenylalanine in steps 6 and 8 of Example 1. The first compound of Scheme 6 was obtained by this route.

Step 2: A solution of tetrakis(triphenylphosphine)palladium (6 mg) in dioxane (3 mL) was prepared. This solution (0.3 mL) was added to the compound from step 1 (5 mg), followed by the addition of hexamethylditin (16 μL). The solution was cooled to −78° C., was pumped under high vacuum, then nitrogen was let into the reaction vessel and then the mixture was allowed to warm to room temperature. The mixture was then heated at 50° C. for 20 minutes, then cooled to room temperature. Ethyl acetate was added. The mixture was washed with 25% ammonium acetate, then brine. The organic layer was dried over magnesium sulfate and filtered, and the solvent was evaporated under vacuum. The residue was purified by silica gel chromatography using 3% methanol in dichloromethane, which contained 0.5% triethylamine.

Step 3: To the product of step 2 (1 mg) in DMF (300 μL) was added 0.2M pH 7 phosphate buffer (38 μL), Chloramine T (27 μL of a 10 mg/1 mL water solution) and a Na¹²⁵I (2.5 mCi) solution in water (50 μL). The mixture was stirred 30 minutes at room temperature, then 2M NaHSO₃ (100 μL) was added. The residue was purified by reverse phase chromatography (C₁₈ column) using 72/28 methanol/25 mM NH₄OAc (the 25 mM NH₄OAc contained 5% methanol).

Step 4: To the compound from step 3 was added chloroform (2 mL) and bromotrimethylsilane (1 mL). The mixture was stirred overnight. The solvent was then evaporated under vacuum and coevaporated with chloroform (2×) followed by coevaporation with methanol (3×). Purification with reverse phase HPLC gave the title compound, labeled with ¹²⁵I, which coeluted with the unlabelled iodine compound.

M.S. (ESI) m/z 908 (M−H)⁻. This is the mass of the cold (unlabelled) compound obtained by treatment of the compound of step 1 as in step 12 of Example 1.

### Example 10

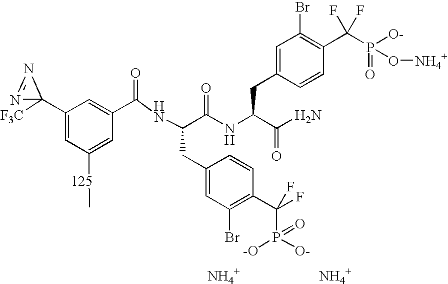

Step 1: Using the same procedure as in example 1, steps 1 to 11, the first compound shown in Scheme 6 (X═Br) was obtained. The procedure of Example 9, steps 2, 3 and 4, was then followed, yielding the compound shown above. The ¹²⁵I-labelled compound eluted on reverse phase HPLC with the nonlabelled compound from example 1.

### Example 11

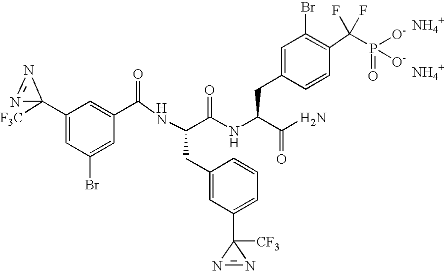

### Diammonium 3-bromo-N-(3-bromo-5-(3-(trifluoromethyl)-3H-diaziren-3-yl)benzoyl)-5-(3-(trifluoromethyl)-3H-diaziren-3-yl)-L-phenylalanyl-3-bromo-4-(difluoro(phosphonato)methyl)-L-phenylalaninamide

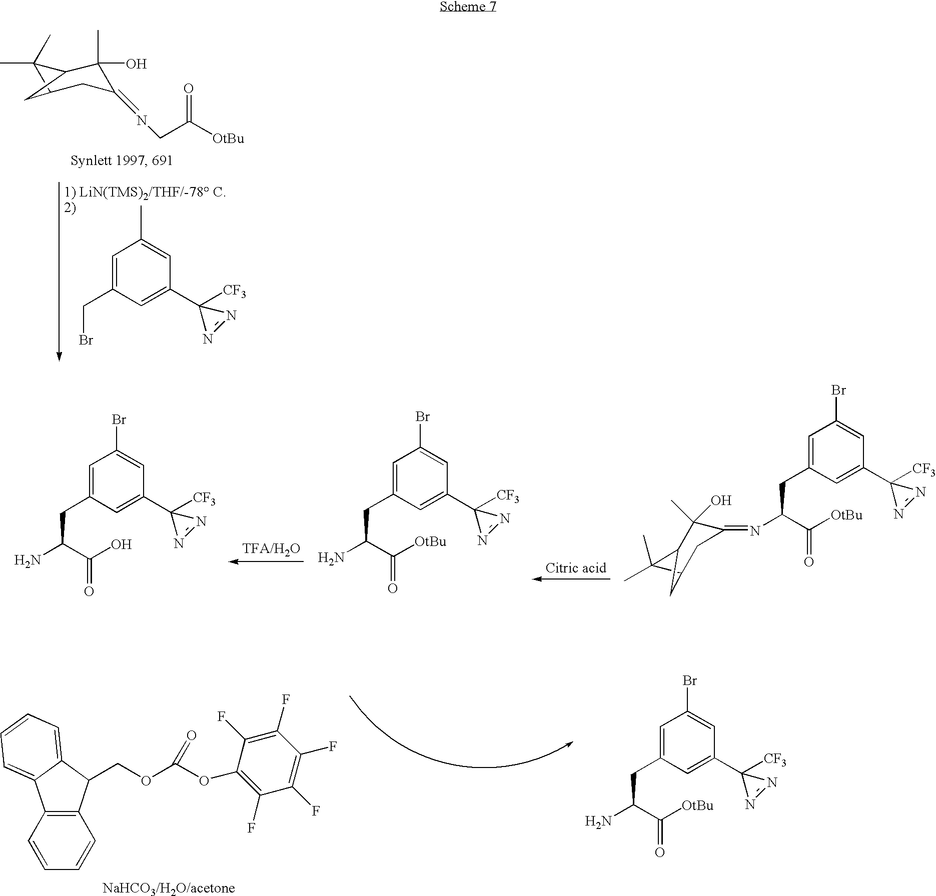

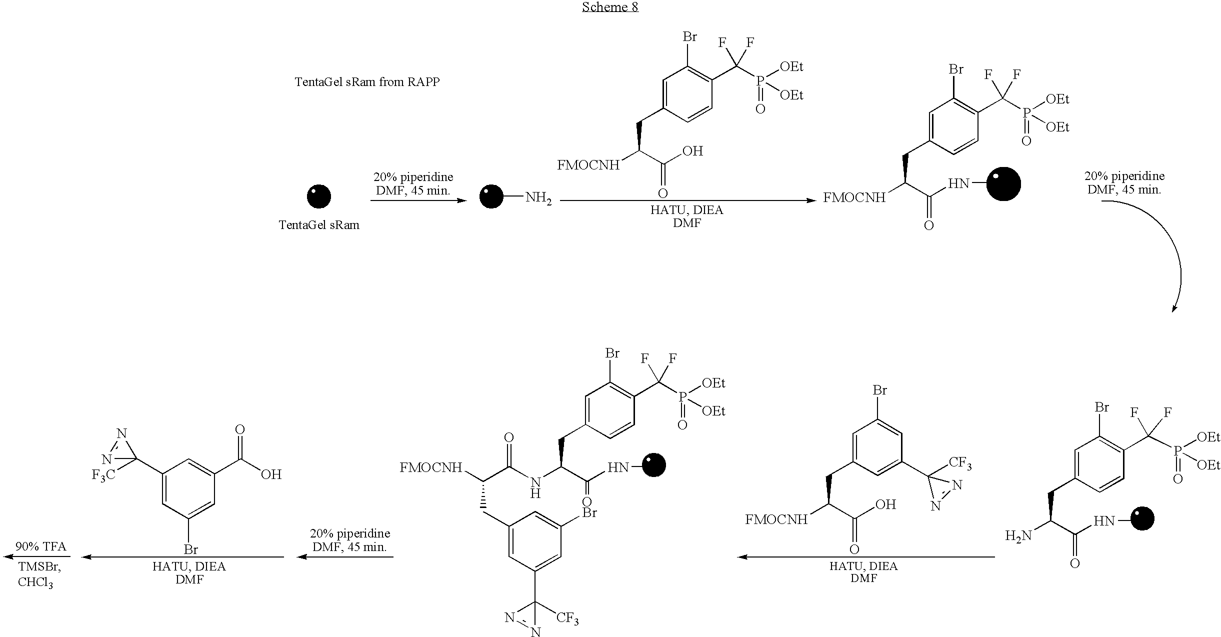

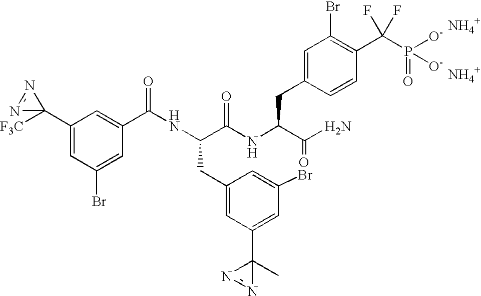

### See Scheme 7: 3-bromo-N-((9H-fluoren-9-ylmethoxy)carbonyl)-5-(3-(trifluoromethyl)-3H diazirin-3-yl)-L-phenylalanine

The same procedure was used as in example 1, Scheme 1, using 3-(3-bromo-5-(bromomethyl)phenyl)-3-(trifluoromethyl)-3H-diazirene instead of diethyl (2-bromo-4-(bromomethyl)phenyl)(difluoro)methylphosphonate in Step 1. The compound that was obtained (280 mg) was stirred in THF (3 mL) and 15% citric acid (3 mL) for 5 days. The solvent was then evaporated. The aqueous portion was washed with diethyl ether (2×) and then made basic with potassium carbonate. It was extracted with ethyl acetate, dried over magnesium sulfate, and filtered, and the solvent was evaporated under vacuum. 104 mg of the trifluoroacetate salt of the obtained compound was dissolved in water (1.5 mL), then sodium bicarbonate (52.5 mg) was added. Acetone (2 mL) was then added followed by addition of a solution of 9H-fluoren-9-ylmethyl pentafluorophenyl carbonate (121 mg) in dioxane (1.5 mL). The resulting solution was stirred at room temperature overnight. The solvent was evaporated under vacuum. The residue was diluted with aqueous sodium bicarbonate and washed with diethyl ether. It was then acidified with 6 N HCl and extracted with ethyl acetate, washed with brine, dried over magnesium sulfate, and filtered. The solvent was evaporated under vacuum. Purification by silica gel chromatography afforded the compound named at the beginning of this paragraph.

Scheme 8: The same procedure was used as in example 1, Scheme 2, Steps 5-9, except that 3-bromo-N-((9H-fluoren-9-ylmethoxy)carbonyl)-5-(3-(trifluoromethyl)-3H-diaziren-3-yl)-L-phenylalanine obtained in Scheme 7 (above) was substituted for 3-bromo-4-((diethoxyphosphoryl)(difluoro)methyl)-N-((9H-fluoren-9-ylmethoxy)carbonyl)-L-phenylalanine in the second amino acid coupling step (steps 8 and 9) of Example 1. This product was then reacted with 3-bromo-5-(3-(trifluoromethyl)-3H-diaziren-3-yl)benzoic acid instead of 3-iodo-5-(3-(trifluoromethyl)-3H-diaziren-3-yl)benzoic acid according to the method of Example 1, steps 10-12, to yield the title compound.

M.S. (ESI) m/z 997.3 (M−H)⁻.

### Labelling Studies Using the PTP1B Photoprobes

Materials

MEM (with Earle's salts and L-glutamine), fetal bovine serum (FBS), penicillin-streptomycin, Dulbecco's phosphate buffered saline (PBS) and Complete protease inhibitor tablets were obtained from Gico-BRL (Rockville, Md.). Anti-PTP1B antibody was purchased from Transduction Laboratories. Mouse anti-FLAG antibody was obtained from Sigma (Cat. No. F3165). Protein A-Sepharose (17-0974-01) was from Amersham. Lowry protein determination reagents were purchased from Bio-Rad (DC). DMH, a DTT substitute was synthesized following the procedure outlined by Singh and Whitesides (33). N-benzoyl-L-glutamyl-[4-phosphono(difluoromethyl)]-L-phenylalanyl-[4-phosphono(difluoromethyl)]-L-phenylanalininamide {alternatively named N-benzoyl-L-α-glutamyl-4-[difluoro(phosphono)methyl]-L-phenylalanyl-4-[difluoro(phosphono)methyl]-L-phenylalaninamide} (BzN-EJJ-amide) was synthesized as previously described (28). All other reagents were from Sigma.

Protein Tyrosine Phosphatases

Protein tyrosine phosphatases used in this study were expressed and purified using published procedures, as follows: (a) Flag-PTP1B (residues 1-320 as a flag fusion), Huyer et al. (29). (b) Flag-TCPTP (residues 1-281 as a flag fusion), Asante-Appiah et al. (30). (c) Flag-CD45 (residues 556-1268 as a flag fusion), Wang et al. (31). (d) Mutant C215S-PTP1B (residues 1-320 as a gst fusion), Skorey et al. (28). (e) Infected SF9 cell lysate expressing full-length PTP1B (as a flag fusion), Cromlish et al. (25). For studies using H₂O₂-inactivated Flag-PTP1B, 100 μl of 2 μg/ml PTP1B was incubated with 100 μl of 1 mM or 20 mM H₂O₂ for 15 min to obtain 50% or 100% inactivated enzyme respectively as determined by the enzyme kinetic assay.

Cell Culture

Human hepatoma cells (HepG2) were obtained from American Type Culture Collection and maintained in regular MEM containing 10% FBS and 1% antibiotics at 37° C., 5% CO2. For photoprobe experiments, cells (2×10⁴) were grown until 80% confluence in 6-well plates and serum starved for 14 hours by incubating in MEM with 1% antibiotics without FBS.

Enzyme Kinetic Assay

Kinetic measurements were performed using a Spectromax Gemini fluorescent plate reader (Molecular Devices, Sunnyvale, Calif.) in a 96-well plate format. Briefly, to 170 μl of BisTris buffer (50 mM BisTris, with and without 150 mM NaCl, 2 mM EDTA, 5 mM DMH, pH 6.3) containing 0-100 μM DiFMUP with or without 0.1% human serum albumin was added 10 μl of 10 or 50 μM photoprobe in water in a three-fold serial dilution. The reaction was initiated by the addition of 20 μl of 0.04 μg/ml Flag-PTP1B, Flag-TCPTP (0.04 μg/ml) or Flag-CD45 (0.01 μg/ml) in BisTris buffer containing 20% glycerol and 0.01% Triton X-100. The conversion of DiFMUP to DiFMU was monitored continuously for 5 min with excitation at 360 nm and emission at 450 nm (cutoff filter at 435 nm). IC50 values were calculated by nonlinear regression fit of the initial rates versus inhibitor concentration obtained using 10 μM DiFMUP using the Softmax Pro software (version 3.11, Molecular Devices). Ki values were determined by non-linear regression fit of the initial rates versus substrate concentration at various inhibitor concentrations using the kinetic equation software from Grafit software (Erithacus Software).

Photolabeling for LC-MS Analysis

All MS data were acquired using a Micromass Q-ToF Ultima mass spectrometer (Manchester, UK) fitted with a Z-spray electrospray ion source. Samples were delivered to the mass spectrometer using an Agilent 1100 capillary-LC (Palo Alto, Calif.). The LC-MS conditions were the same as previously published.

Photolabeling of Purified Enzyme Using Radioactive Photoprobes

All photolabeling experiments with purified enzymes or SF9 cell lysate overexpressing PTP1B were conducted in 96-well polystyrene plates in 50 μl BisTris buffer (50 mM BisTris pH 6.3, 2 mM EDTA, 1 mM DMH) containing enzyme (in BisTris buffer with 20% glycerol, 0.01% Triton X-100) or lysate and Example 9 (in water) or Example 1 (in water) at various concentrations and shaken for 1 min at room temperature. For competition studies, BzN-EJJ-AMIDE at various concentrations was added prior to the addition of the probes. The samples were then placed on ice for 5 min and irradiated with the 40 watt lamp at the standard 10 cm distance for 15 minutes. The irradiated samples were quenched with SDS sample buffer and SDS-PAGE electrophoresis was conducted using 10% Tris-glycine gels (27). Autoradiography of the dried, coommassie-stained gel was done using Phosphorimaging (Molecular Devices) or by x-ray film (BioMax MS with intensifier screen, Kodak) and quantified by densitometry.

Photolabeling of HEPG2 Cells Using Radioactive Photoprobes

HEPG2 cells were grown to ˜80% confluency in 6-well dishes. Media was removed and washed twice with Hanks-Hepes buffer (15 mM Hepes). For HepG2 lysate studies, the cells were lysed on ice for 15 minutes using 1 ml lysate buffer (50 mM Tris pH 7.4, 150 mM NaCl, 1% Triton X-100, 5 mM EDTA, 5 mM DTT, Complete™ protease inhibitor tablet). For HepG2 whole cell studies, 1 ml Hanks-Hepes buffer was added to the washed cells in the 6-well plates. The compound of Example 1 at various concentrations was added to either the lysate buffer cells or Hanks-Hepes whole cell and incubated for various times at room temperature prior to irradiation on ice with the 40 watt lamp for 15 minutes. For competition studies with inhibitors, compounds at various doses were incubated for 30 min with the HEPG2 whole cells or 1 min with the HEPG2 lysate prior to addition of the photoprobe. The cells were then placed on ice and irradiated with the 40 watt lamp for 15 min. The HepG2 whole cells were then lysed and the lysate centrifuged at 14,000 rpm for 15 min at 4° C. The supernatant was pre-cleared by the addition of 100 μl (50% slurry in lysis buffer) of Protein A sepharose beads with rotation for 10 min at 4° C. followed by centrifugation at 14,000 rpm for 10 min at 4° C. A stock of HepG2 lysate was also obtained from a T175 confluent flask. The protein concentration of the lysates was determined by the method of Lowry (detergent compatible).

PTP1B was immunoprecipitated from HepG2 cell lysates (1 ml of 1 mg/ml) using 3.5 ug of anti-PTP1B antibody (BDT) overnight at 4° C. with rotation. Prewashed Protein G-sepharose beads (100 μL of packed beads) were added for 2 h at 4° C. The beads were washed three times with PBS and protein was eluted using SDS sample buffer and heated at 95° C. for 5 min. SDS-PAGE was carried out by the method of Laemmli (27) on 10% polyacrylamide gels in 25 mM Tris base pH 8.3, 192 mM glycine, 0.1% SDS at 140 V constant voltage. For autoradiogram detection of radioactivity, the gels were dried and exposed to BioMax MS film with an intensifying screen at −80° C. Protein bands were quantified using a scanning densitometer (Bio-Rad).

Photochemistry of the Photoprobes.

The photoprobes that were made in this invention are structurally related to the tripeptide BzN-EJJ-AMIDE (see structure below), a selective, potent inhibitor of both PTP1B and TCPTP1B (23). Examples 1 and 9 are dipeptides that have been modified chemically by incorporation of the photolabile trifluoromethylphenyldiazirene group (24) into a dipeptide that is structurally similar to BzN-EJJ-AMIDE. Example 1 also includes a 3-bromo substituent on the two 4-difluoro(phosphono)methylphenyl rings. The presence of the bromine substituent ortho to the difluoro(phosphono)methyl group on similar non-brominated compounds generally increases the potency by about 100-fold. These probes were tested for their usefulness in labeling PTP1B. The photoprobes are stable in the dark, and can be activated using a low power lamp (40 W, Philips). The photochemical conditions are not expected to affect the integrity of proteins and cells.

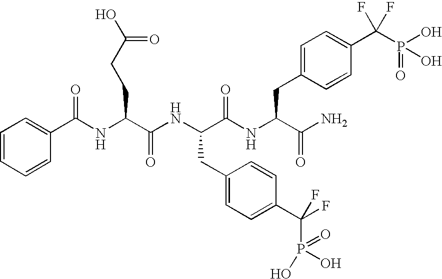

### BzN-EJJ-AMIDE

A time course for photolysis of the probes in water was determined by measuring product formation by HPLC-MS. Irradiation of the probe in an aqueous medium resulted in a single photolysis product with a mass equivalent to the loss of N₂ and subsequent reaction of H₂O with the carbene intermediate. If the probe was irradiated in methanol, a single product was observed with an increased mass equivalent to the loss of N₂ and reaction of the carbene intermediate with CH₃OH. Based on the time course of photolysis of an aqueous probe with a 40 W lamp, t_{1/2} values of about 2 min were measured using the 40 W lamp. When a 450 W lamp was used, the t_{1/2} was reduced to less than 30 sec. The stronger 450 W lamp also decreased or destroyed PTP1B enzyme activity, and was not used for enzyme labeling experiments.

The quantum yield of the photoprobes was determined from the number of photons absorbed and the number of moles of photolysis product that were formed. The number of photons absorbed was determined by chemical actinometry, using the method of Parker (26). The moles of irradiated product produced over time was measured by measuring the absorbance at 345 and 350 nm for Examples 9 and 1 respectively. The quantum yield was calculated to be 0.83 and 0.74 for Examples 9 and 1 respectively.

Labelling of Purified PTP1B Using Non-radioactive Photoprobes

The non-irradiated probes were found to be competitive, reversible inhibitors of PTP1B with Ki values of 5 and 0.1 nM for Example 9 and Example 1 respectively. A summary of the IC50 data obtained with the photoprobes treated with PTP1B, TCPTP and CD45 in the dark are summarized in Table 1. The photoprobes were equally potent on PTP1B and TCPTP and showed high selectivity over CD45. The IC50 for Example 1 was shifted 7 fold in the presence of 0.1% protein whereas the potency of Example 9 was not significantly affected by protein.

TABLE 1
IC₅₀ of Photoprobes Example 9 and Example 1

IC50 (nM)

| Enzymes | Example 9 | Example 1 |
|---|---|---|
| Flag-PTP1B(1-320) | 11 | 0.2 |
| Flag-TCPTP | 6 | 0.1 |
| Flag-CD45 | 12600 | 490 |
| Fla-PTP1B + 0.1% HSA | 14 | 1.5 |

To detect photoprobe labeling of the enzyme after irradiation, the probe-enzyme complex was subjected to LC-MS analysis. After irradiation of a 10-fold excess of probe with recombinant PTP1B on ice for 20 min with a 40 W lamp, a 1:1 adduct was observed accounting for ˜8% of the total enzyme. No significant multiple adduct formations were observed. No adduct formation was found without irradiation. The enzyme alone was not affected by irradiation with the 40 W lamp for up to 30 min. There was no appreciable loss of enzyme activity during this time. Furthermore, the probe-enzyme complex before irradiation competed with active-site inhibitors such as the tripeptide BzN-EJJ-AMIDE, suggesting that the probe binds at the active site. The extent of labeling of PTP1B could be increased by repeating the irradiation experiments after removal of the unlabeled probe by gel filtration.

Labeling of Purified Enzyme Using ¹²⁵I-Radiolabelled Photoprobes.

To study the labeling of PTP1B under physiological concentrations that exist in cells and tissues, ¹²⁵I -radiolabeled versions of Examples 9 and 1 were synthesized. Purified PTP1B was incubated with the photoprobe for 5 min and was then irradiated for 20-30 min on ice with a 40 W low UV lamp. The labeled enzyme was detected by phosphorimage (Molecular Devices) or x-ray film (BioMax MS with intensifier screen) from a SDS-PAGE gel after electrophoresis. From a titration of the purified PTP1B and probe, a detection limit for the two probes of Examples 9 and 1 were determined to be about 5 and 0.2 nM respectively, as detected by phosphorimaging. The use of an X-ray film with the Biomax intensifier was found to be 20-50 times more sensitive than phosphorimaging, so that 0.1 nM or less of labeled enzyme could be detected with the photoprobe of Example 1. To determine if the probes were specific for PTP1B in the presence of other proteins, the probes were irradiated with an SF9 cell-line containing overexpressed PTP1B (1% wt/wt protein) (25). Both probes that were tested (Examples 1 and 9) labeled SF9 overexpressed PTP1B. A time-course for irradiation was determined. For probe concentrations less than 20 nM, maximum labeling occurred within 10 min. Preincubation of the probe with purified enzyme for longer than 5 min prior to irradiation did not enhance the labeling. Labeling of both recombinant PTP1B and full-length PTP1B in SF9 lysate for either Example 1 or 9 was observed to compete with a known active-site inhibitor. The probes were specific for PTP1B or TCPTP and did not label CD45. The probes could also label the C215S mutant of PTP1B, and this labeling was observed to compete with the active site inhibitor BzN-EJJ-AMIDE. It was furthermore found that the photoprobes only labelled active enzyme. Thus the probe of Example 1 would not label PTP1B that had been partially or fully inactivated by hydrogen peroxide. PTP1B or the C215S mutant of PTP1B that had been subjected to heat denaturation also could not be labeled by the photoprobes of Example 1.

An estimate of the efficiency of labeling was measured by comparing the maximum signal obtained with 7 nM Example 9 or 7 nM Example 1 with purified Flag-PTP1B and the signal obtained with 7 nM Example 9 or Example 1 after gel electrophoresis of the probe alone. Efficiencies of 9 and 11% were measured for Examples 9 and 1 respectively.

Labeling of Endogenous PTP1B in HEPG2 Cell Lysate

A titration of photoprobe labeling of PTP1B in HEPG2 cell lysate was conducted using 0.04-5 nM of labeled Example 1 and 200 μl of cell lysate (2.5 mg/ml) in salt-containing cell lysate buffer. After immunoprecipitation of PTP1B, the radiogram of the 10% SDS-Page gel of the immunoprecipitation samples showed saturation of the PTP1B around 0.6 nM probe. This corresponded closely to the predicted concentration of PTP1B in the lysate of 1 nM based on 0.25 ng PTP1B/μg total protein. The x-ray film was sensitive enough to detect as little as 0.04 nM of the labeled immunoprecipitated PTP1B. To be certain that the immunoprecipitation efficiency was not affected by the photoprobe labeling, the western signals obtained with PTP1B antibody with HEPG2 cells treated with and without photoprobe and before and after immunoprecipitation were compared. There was no difference in the amount of PTP1B detectable by Western blot analysis before and after immunoprecipitation and between labeled and unlabeled enzyme.

Labeling of Endogenous PTP1B in HEPG2 Whole Cells

The compound of Example 1 at various concentrations was incubated with HEPG2 whole cells for 30 min and was irradiated on ice for 15 min. The cells were then lysed, and labeled PTP1B was measured after immunoprecipitation. The photoprobe labeled PTP1B in the HEPG2 whole cell in a dose-dependent manner, with saturation occurring at around 10 nM. Although this appears to become saturated at a much higher concentrations than the 1 nM that is calculated based on the concentration of PTP1B in 2.5 million cells/ml buffer, this is consistent with the following explanation. The whole cell reduces the PTP1B labeling to ˜10% of that observed with lysed cells, because only about 10% of the photoprobe enters the cell. The 10% cell permeability of the photoprobe was verified by performing a mass balance on the total counts obtained with 5 nM probe and whole cell after washes and immunoprecipitation. By this technique, about 10% of the probe was found to be associated with the cell, and the remaining 90% of the total counts were found in the media before cell lysis. Of the 10% that was associated with the cell, only 1% of the probe labeled the PTP1B. The remainder was found at the solvent front after gel electrophoresis and was assumed to be probe that reacted with water after irradiation and was not bound to the PTP1B. The labeling of PTP1B in the whole cell was completely prevented by addition of 1 μM of non-radioactive photoprobe, presumably due to competitive inhibition of the radiolabelled photoprobe.

Cell viability was measured both by trypan blue exclusion assays and lactate dehydrogenase (LDH) release and found to be <2% compared to lysed cells in all photolysis experiments conducted with HEG2 whole cells. This was below the minimum 10% lysis required to account for the whole cell labeling. This supports that the photolabeling of proteins in HEPG2 cells occurs in intact cells and not from the small amount of proteins released outside the cell.

To optimize the labeling conditions, incubation and irradiation times were varied. With the whole cell, the labeling was found to be time-dependent, requiring 30 min incubation with the probe prior to irradiation to achieve the maximum signal. Cell lysate did not require any significant probe incubation. This provides further evidence that the signal that was measured in the whole cell experiments was from PTP1B in the cell, as the probe needs time to enter the cell. Irradiation time needed to effect the labeling was the same for either whole cell or lysate. About 10 min of irradiation was required for both conditions to maximize the labeling.

Competition with several known PTP1B inhibitors was conducted in HEPG2 whole and lysed cells using 0.4 or 1 nM photoprobe. The IC50 values estimated from these competitive photoaffinity experiments correspond closely to IC50 values estimated from data with purified recombinant PTP1B in the presence of 0.1% human serum albumin and 150 mM salt. This indicates that these inhibitors are cell permeable and can occupy PTP1B inside the cell, resulting in competition with the photoaffinity labeling of PTP1B by the photoprobe.

### REFERENCES CITED

1. DeFronzo, R. A., Bonadonna, R. C., and Ferrannini, E. (1992) Diabetes Care 15, 318-368.
2. Reaven, G. M. (1993) Diabetes Metab Rev. 9 Suppl 1, 5S-12S.
3. Kahn, C. R. (1994) Diabetes 43, 1066-1084.
4. Dadke, S., Kusari, J., and Chernoff, J. (2000) J Biol Chem 275, 23642-23647.
5. Bandyopadhyay, D., Kusari, A., Kenner, K. A., Liu, F., Chernoff, J., Gustafson, T. A., and Kusari, J. (1997) J Biol Chem 272, 1639-1645.
6. Dadke, S., Kusari, A., and Kusari, J. (2001) Mol Cell Biochem 221, 147-154.
7. Seely, B. L., Staubs, P. A., Reichart, D. R., Berhanu, P., Milarski, K. L., Saltiel, A. R., Kusari, J., and Olefsky, J. M. (1996) Diabetes 45, 1379-1385.
8. Wang, X. Y., Bergdahl, K., Heijbel, A., Liljebris, C., and Bleasdale, J. E. (2001) Mol Cell Endocrinol. 173, 109-120.
9. Ding, W., Zhang, W. R., Sullivan, K., Hashimoto, N., and Goldstein, B. J. (1994) Biochem Biophys Res Commun 202, 902-907.
10. Kenner, K. A., Anyanwu, E., Olefsky, J. M., and Kusari, J. (1996) J Biol Chem 271, 19810-19816.
11. Elchebly, M., Payette, P., Michaliszyn, E., Cromlish, W., Collins, S., Loy, A. L., Normandin, D., Cheng, A., Himms-Hagen, J., Chan, C. C., Ramachandran, C., Gresser, M. J., Tremblay, M. L., and Kennedy, B. P. (1999) Science 283, 1544-1548.
12. Iversen, L. F., Andersen, H. S., Moller, K. B., Olsen, O. H., Peters, G. H., Branner, S., Mortensen, S. B., Hansen, T. K., Lau, J., Ge, Y., Holsworth, D. D., Newman, M. J., and Hundahl Moller, N. P. (2001) Biochemistry 40, 14812-14820.
13. Andersen, H. S., Iversen, L. F., Jeppesen, C. B., Branner, S., Norris, K., Rasmussen, H. B., Moller, K. B., and Moller, N. P. (2000) J Biol Chem 275, 7101-7108.
14. Larsen, S. D., Barf, T., Liljebris, C., May, P. D., Ogg, D., O'Sullivan, T. J., Palazuk, B. J., Schostarez, H. J., Stevens, F. C., and Bleasdale, J. E. (2002) J Med Chem 45, 598-622.
15. Wang, Q., Huang, Z., Ramachandran, C., Dinaut, A. N., and Taylor, S. D. (1998) Bioorg Med Chem Lett 8, 345-350.
16. Malamas, M. S., Sredy, J., Moxham, C., Katz, A., Xu, W., McDevitt, R., Adebayo, F. O., Sawicki, D. R., Seestaller, L., Sullivan, D., and Taylor, J. R. (2000) J Med Chem 43, 1293-1310.
17. Malamas, M. S., Sredy, J., Gunawan, I., Mihan, B., Sawicki, D. R., Seestaller, L., Sullivan, D., and Flam, B. R. (2000) J Med Chem 43, 995-1010.
18. Iversen, L. F., Andersen, H. S., Branner, S., Mortensen, S. B., Peters, G. H., Norris, K., Olsen, O. H., Jeppesen, C. B., Lundt, B. F., Ripka, W., Moller, K. B., and Moller, N. P. (2000) J Biol Chem 275, 10300-10307.
19. Artursson, P. and Borchardt, R. T. (1997) Pharm Res 14, 1655-1658.
20. Dorman, G. and Prestwich, G. D. (2000) Trends Biotechnol. 18, 64-77.
21. Dorman, G. and Prestwich, G. D. (1994) Biochemistry 33, 5661-5673.
22. Weber, P. J. and Beck-Sickinger, A. G. (1997) J Pept. Res 49, 375-383.
23. Desmarais, S., Friesen, R. W., Zamboni, R., and Ramachandran, C. (1999) Biochem J 337 (Pt 2), 219-223.
24. Brunner, J., Senn, H., and Richards, F. M. (1980) J Biol Chem 255, 3313-3318.
25. Cromlish, W. A., Payette, P., and Kennedy, B. P. (1999) Biochem Pharmacol 58, 1539-1546.
26. Parker, C. A. (1953) Proc. R. Soc. London Ser. A 220, 104.
27. Laemmli, U. K. (1970) Nature (London) 227, 680-685.
28. Skorey, K. I., Kennedy, B. P.; Friesen, R. W., Ramachandran (2001) C. Anal. Bioche 291, 269-278).
29. Huyer, G., Liu, S., Kelly, J., Moffat, J., Payette, P., Kennedy, B., Tsaprailis, G., Gresser, M. J., and Ramachandran, C. (1997) J Biol. Chem. 373, 843-851
30. Asante-Appiah, E., Ball, K., Bateman, K., Skorey, K., Friesen, R., Desponts, C., Payette, P., Bayly, C., Zamboni, R., scapin, G., Ramachandran, C., and Kennedy, B. P. (2001) J Biol. Chem. 376, 36036-36043
31. Wang, Q., Scheigetz, J., Gilbert, M., Snider, J., and Ramachandran, C. (1999) Biochem. Biophys Acta 1431, 14-33
32. Cromlish, W. A., Payette, P., and Kennedy, B. P. (1999) Biochem Pharmacol 58, 1539-1546.
33. Singh, R., and Whitesides, G. M. (1991) J Org. Chem. 56, 2332-2337.

What is claimed is:

1. A compound having the formula I, or a salt thereof: 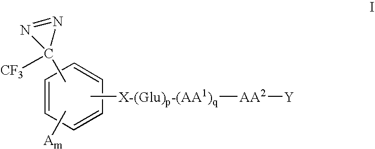
wherein Glu is a glutamic acid residue wherein the gamma-carboxy group of said glutamic acid residue is a free carboxylic acid or a C₁₋₃ alkyl ester;
AA¹ is an amino acid residue having the formula 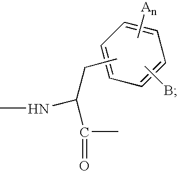
AA² is an amino acid residue having the formula 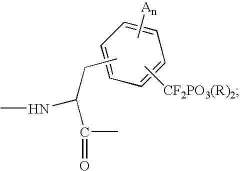
X is a difunctional group selected from CH₂ and carbonyl;
Y is selected from the group consisting of OH and NR₂;
Each R is independently selected from the group consisting of H and C₁₋₆ alkyl, said C₁₋₆ alkyl being linear or branched;
A is a substituent selected from the group consisting of CH₃, CF₃, and halogen;
Each B is a substituent selected from the group consisting of H, —CF₂PO₃(R)₂ and 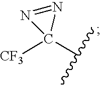
m is O, 1,or2;
each n is independently 0, 1, or 2;
p is 0, 1, or 2; and
q is 0, 1, or 2.

2. A compound of claim 1, wherein
Glu is a glutamic acid residue, wherein the gamma carboxy group of said glutamic acid residue is a free carboxylic acid or a methyl ester;
m is O or 1;
each n is independently 0 or 1; and
p is 0 or 1.

3. A compound of claim 2, wherein
Each A is a halogen independently selected from F, Cl, Br, and I;
Each group B is a substituent selected from —CF₂PO₃H₂ and 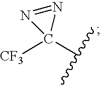
Y is NH₂;
R is H; and
q is 0 or 1.

4. A compound of claim 3, wherein Glu is a glutamic acid residue in which the gamma carboxy group is a free carboxylic acid residue.

5. A compound of claim 1, wherein AA¹ and AA² are each phenylalanine residues, wherein the substituents on the phenyl ring of said phenylalanine residues are as defined in claim 1.

6. A compound of claim 1, wherein AA¹ and AA² are amino acid residues having the formula 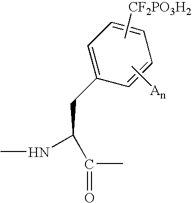
Wherein each A is independently selected from the group consisting of Br and I;
m is 0 or 1;
each n is independently 0 or 1;
p is 0 or 1;
q is 0 or 1; and
Y is NH₂.

7. A compound according to claim 1, wherein AA¹ is an amino acid residue having the formula: 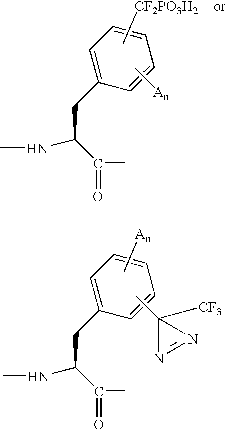
wherein Glu is a glutamic acid residue, the gamma-carboxy group of said glutamic acid residue being a free carboxylic acid or methyl ester;
AA² is a phenylalanine residue of the formula 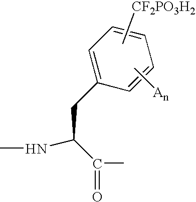
Each A is independently selected from the group consisting of Br and I;
Y is —NH₂;
R is H;
m is 0 or 1;
each n is independently 0 or 1;
p is 0 or 1; and
q is 0 or 1.

8. A compound of claim 7, wherein Glu is a glutamic acid residue in which the gamma carboxy group is a free carboxylic acid residue.

9. A compound of claim 1, or a salt thereof, having a structural formula selected from the group consisting of: 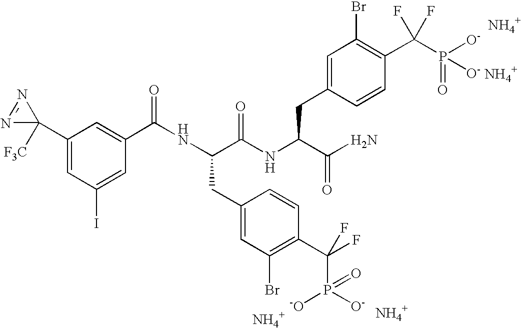 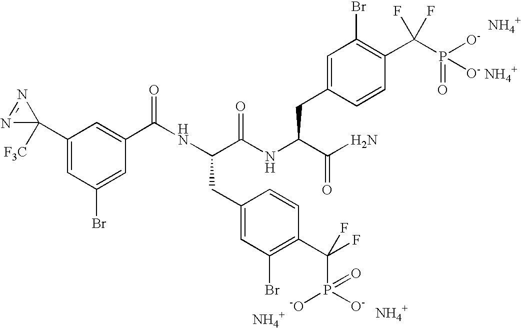 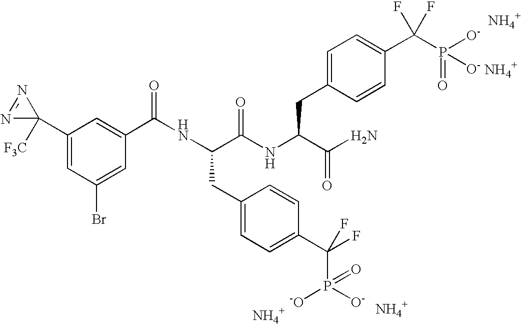 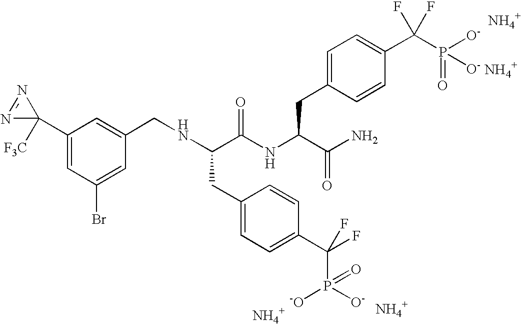 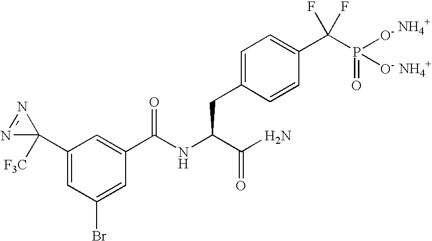 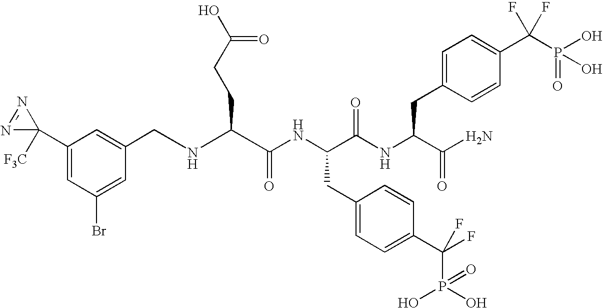 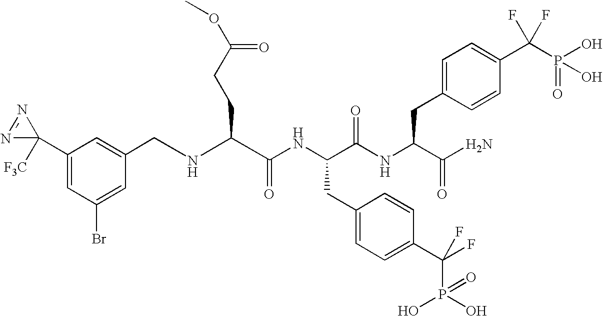 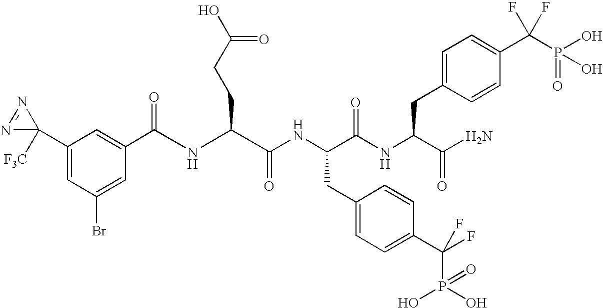 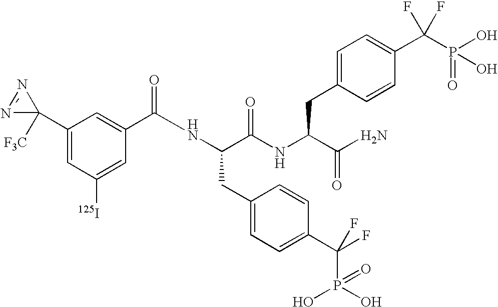 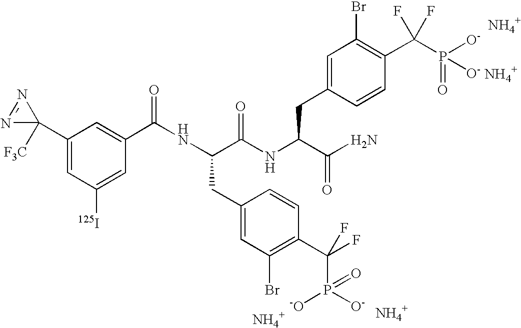
and [chemical structure]
